(19) United States
(12) Patent Application Publication
(10) Pub. No.: US 2026/0114969 A1
(43) Pub. Date: Apr. 30, 2026

(54) SOFT DENTURE RELINING MATERIAL

(71) Applicant: GC America Inc., Alsip, IL (US)

(72) Inventors: Futoshi FUSEJIMA, Alsip, IL (US); Alec HART, Chicago, IL (US); Megan BURKE, Chicago, IL (US)

(73) Assignee: GC America Inc., Alsip, IL (US)

(21) Appl. No.: 19/373,874

(22) Filed: Oct. 30, 2025

Related U.S. Application Data
(60) Provisional application No. 63/714,477, filed on Oct. 31, 2024.
(60) Provisional application No. 63/798,106, filed on May 1, 2025.

(51) Int. Cl. A61C 13/07; A61K 6/889

(52) U.S. Cl. CPC A61C 13/0025; A61K 6/889

(57) ABSTRACT

A composition for a soft relining material, including: 10 to 70% by weight of a methacrylate monomer having at least one unsaturated double bond; 30 to 80% by weight of a plasticizer; 5% to 20% by weight of a solvent; up to 2% by weight of a stabilizer; up to 0.5% by weight of an inhibitor; and 0.25 to 5% by weight of an accelerator.

This application claims benefit of U.S. provisional application Nos. 63/714,477 filed Oct. 31, 2024 and 63/798,106 filed May 1, 2025, the contents of which are incorporated herein by reference.

### BACKGROUND

### 1. Technical Field

The present disclosure relates to a soft relining material, and more particularly a soft relining material capable of bonding with resin composite base (cross-linked multi-functional resin polymer) dentures (i.e. three dimensional (“3D”) printed resin dentures) and acrylic base dentures.

### 2. Description of Related Art

For many years, dentures have been made mostly of acrylic materials. Soft reliners are commonly used with dentures to provide a softer coating on a base of the dentures so that the dentures are more comfortable for the wearer. Soft reliners are commonly made of a silicone material (“silicone reliner”) or a powder-liquid mixture. Silicone reliners tend to cost more than traditional liquid-powder reliners. Silicone reliners tend to bond well with all denture types, while traditional liquid-powder reliners tend to only bond to acrylic based dentures.

In a traditional liquid-powder reliner, the liquid contains an alcohol (e.g. ethanol) and a plasticizer (e.g. dibutyl phthalate) and the powder contains an acrylic polymer (e.g. polyethyl methacrylate). When the liquid and powder are mixed, the liquid swells and unwinds the polymer chains in the powder and forms a putty-like material. When the putty-like material contacts a base of an acrylic based denture, the polymers of the base are swelled and unwound, allowing the putty-like material to adhere to the acrylic based denture.

In contrast to acrylic based dentures, 3D printed dentures have a resin composite base made of multi-functional resin (i.e. dimethacrylate). The dimethacrylate components provide a high degree of crosslinking in the denture base polymer chains resulting in a durable material. However, the traditional liquid-powder reliner fails to entangle with the multi-functional resin (i.e. dimethacrylate) of the 3D printed denture base resulting a poor adhesion between the liquid-powder reliner and the 3D printed denture base. Instead, silicone reliners are currently used to bond to the 3D printed denture base.

With the increase in technology, the use of 3D printers to create dentures has grown. Because the 3D printed dentures commonly have a base made of a cross-linked multi-functional resin instead of an acrylic material, silicone reliners for the 3D printed dentures are often used instead of traditional liquid-powder reliners. Therefore, a wearer of 3D printed dentures incurs a higher cost for a soft reliner.

There is a need for a liquid-powder reliner that effectively bonds to both 3D printed denture bases (cross-linked multi-functional resin polymer bases) and acrylic denture bases while also being low cost.

### SUMMARY

The present disclosure may solve the above problems and other problems.

Provided is a liquid-powder reliner including an acrylic monomer for allowing the liquid-powder reliner to be capable of adhering to both 3D printed denture bases (cross-linked multi-functional resin polymer bases) and acrylic denture bases.

According to an aspect of the present disclosure, a composition for a soft relining material includes: 10 to 70% by weight of a methacrylate monomer having at least one unsaturated double bond; 30 to 80% by weight of a plasticizer; 5% to 20% by weight of a solvent; up to 2% by weight of a stabilizer; up to 0.5% by weight of an inhibitor; and 0.25 to 5% by weight of an accelerator.

The plasticizer may include at least one of of dibutyl phthalate, acetyl tributyl citrate, benzyl salicylate, trimelitic acid esters, fatty acid esters, dicarboxylic acid esters, acetic acid esters, maleic acid esters, fumaric acid esters, and citric acid esters.

The plasticizer may include a mixture of acetyl tributyl citrate and benzyl salicylate.

The plasticizer may include acetyl tributyl citrate.

The plasticizer may include benzyl salicylate.

The plasticizer may be 65% by weight.

The solvent may be ethanol.

The stabilizer may be 2,4-dihydroxybenzophenone.

The stabilizer may be an ultra violet (UV) stabilizer.

The stabilizer may be 1% by weight.

The inhibitor may be moquinol (MEHQ).

The inhibitor may be butylated hydoxytoluene (BHT).

The inhibitor may be 0.1% by weight.

The accelerator may be dimethylparatoluidine.

### DETAILED DESCRIPTION

Reference will now be made in detail to non-limiting example embodiments of the present disclosure.

In the present disclosure, the term “at least one of A, B or C” may be interpreted as “only A,” “only B,” “only C,” “A and B,” “A and C,” “B and C,” or “A, B, and C.”

Provided is a low cost liquid-powder reliner that effectively bonds to both 3D printed denture bases (cross-linked multi-functional resin polymer bases) and acrylic denture bases.

### [Description of the Liquid]

According to one or more embodiments, the liquid may include an acrylic monomer, an aliphatic acid ester-based plasticizer, a solvent, a stabilizer, and an amine accelerator. More specifically, according to one or more embodiments, the liquid may include 10 to 70% by weight of a methacrylate monomer having at least one unsaturated double bond, 30 to 80% by weight of a plasticizer, 5% to 20% by weight of a solvent, up to 2% by weight of a stabilizer, up to 0.5% by weight of an inhibitor, and 0.25 to 5% by weight of an accelerator. Before the liquid and the powder are mixed, the liquid may be a volatile or non-volatile material with a low viscosity. The viscosity of the liquid may be 10-3 Pa-s. The liquid may have a viscosity similar to water. The liquid may be a light-translucent yellow color. According to one or more embodiments, the liquid may be stored in a temperature of 20-25° C. The liquid is not limited to the color or may not be stored in the temperature states above.

According to one or more embodiments, the acrylic monomer may be methacrylate having at least one unsaturated double bond that swells polymer chains in both 3D printed denture bases (cross-linked multi-functional resin polymer bases) and acrylic denture bases. According to one or more embodiments, isobutyl methacrylate is used as the methacrylate. According to other embodiments, the methacrylate may include monomers of at least one of methyl methacrylate, ethyl methacrylate, isopropyl methacrylate, 2-hydroxyethyl methacrylate, 3-hydroxypropyl methacrylate, 2-hydroxy-1,3-dimethacryloxypropane, t-butyl methacrylate, n-butyl methacrylate, butoxyethyl methacrylate, hydroxypropyl methacrylate, tetrahydrofurfuryl methacrylate, glycidyl methacrylate, 2-methoxyethyl methacrylate, 2-ethylhexyl methacrylate, benzyl methacrylate, ethylene glycol methacrylate, diethylene glycol dimethacrylate, 1,3-butylene glycol dimethacrylate, 1,4-butylene glycol dimethacrylate, or polyoxytetraethylene glycol dimethacrylate. According to one or more embodiments, the liquid in the liquid-powder reliner may include 10% to 70% by weight of the acrylic monomer having at least one saturated double bond. A minimum amount of 10% by weight of the acrylic monomer may be required such that the liquid-powder reliner can polymerize and adhere the 3D printed denture bases (cross-linked multi-functional resin polymer bases) and acrylic denture bases. When there is more than 70% by weigh of the acrylic monomer, the plasticizers may not provide enough softness for the putty-like consistency of the liquid-powder reliner after the liquid and the powder are mixed.

According to one or more embodiments, the aliphatic acid ester-based plasticizer may be at least one of trimelitic acid esters, fatty acid esters, dicarboxylic acid esters, acetic acid esters, maleic acid esters, fumaric acid esters, or citric acid esters. According to one or more embodiments, the aliphatic acid ester-based plasticizer of the liquid may include at least one of acetyl tributyl citrate or the benzyl salicylate. According to other embodiments, the trimellitic acid esters include at least one of tri-2-ethylhexyl trimelitate; the dicarboxylic acid esters include dimethyl adipate, diisobutyl adipate, dibutyl adipate, diisobornyl adipate, di-2-ethylhexyl adipate, diisodecyl adipate, diethyl diglycol adipate, dibutyl diglycol adipate, di-2-ethylhexyl azelate, dimethyl Sebacate, dibutyl Sebacate di-2-ethylhexyl Sebacate, the fatty acid esters include methylacetyl ricinolate, and epoxidized Soybean oil; the acetic acid esters include glyceryl triacetate and 2-ethylhexyl acetate; the maleic acid esters include diethyl maleate, dibutyl maleate, and di-2-ethylhexyl maleate; the fumaric acid esters include dibutyl fumarate and di-2-ethylhexyl fumarate; and the citric acid esters include trimethyl citrate, triethyl citrate, tripropyl citrate, tributyl citrate, triisopropyl citrate, dibutyl phthalate, or triisobutyl citrate. The aliphatic acid ester-based plasticizers may be used singly or in admixture and may be a safety plasticizer that may not act as an endocrine disruptor to living bodies. The plasticizer may be a mixture of tributyl citrate and benzyl salicylate, or may singly be one of tributyl citrate or benzyl salicylate. According to one or more embodiments, the liquid in the liquid-powder reliner may include 30% to 80% by weight of the aliphatic acid ester-based plasticizer. When there is less than 30% by weight of the aliphatic acid ester-based plasticizer, the putty-like consistency of the liquid-powder reliner after the liquid and the powder are mixed may not be soft, resulting in discomfort for the wearer. When there is more than 80% by weight of the aliphatic acid ester-based plasticizer, there may not be enough of the acrylic monomer to polymerize and adhere both a 3D printed denture base (cross-linked multi-functional resin polymer bases) and an acrylic denture base. Preferably, the liquid in the liquid-powder reliner may include between 60% and 65% by weight of the plasticizer. According to one or more embodiments, ethanol may be used as a solvent. According to one or more embodiments, the solvent may be at least one of acetone, alcohol, or water. According to one or more embodiments, the liquid in the liquid-powder reliner may include 5% to 20% by weight of the solvent. The solvent may be configured to penetrate and swell the acrylic polymer of the powder component in the liquid-powder reliner. If there is less than 5% of the solvent in the liquid-powder reliner, the swelling of the polymers chains in the denture base by the acrylic monomer may be delayed and the liquid-powder reliner may become too hard before applying it to any denture base, regardless of whether it is a 3D printed denture base or an acrylic denture base, for use. If there is more than 20% of the solvent in the liquid-powder reliner, the durability of the putty-like consistency of the liquid-powder reliner after, the liquid and the powder are mixed, may be reduced.

According to one or more embodiments, the plasticizer and the solvent may be mixed into one solution. In a state in which the plasticizer and the solvent are mixed into one solution, the liquid in the liquid-powder reliner may include 5% to 85% by weight of the mixture of the plasticizer and the solvent.

According to one or more embodiments, 2,4-dihydroxybenzophenone may be used as a stabilizer. According to one or more embodiments, the stabilizer may an ultra violet (UV) stabilizer configured to protect the reliner from long-term breakdown caused by UV radiation. For example, the stabilizer may be at least one of mono methyl ether of hydroquinone (MEHQ), butylated hydoxytoluene (BHT), or Hydroquinone (HQ). According to one or more embodiments, the liquid in the liquid-powder reliner may include up to 2% by weight of the stabilizer. If more than 2% by weight of the stabilizer is in the liquid-powder reliner, a setting time of the reliner, after the liquid and the powder are mixed, may be prolonged such that the reliner is set too slowly. Preferably, the liquid in the liquid-powder reliner may include 1% by weight of the stabilizer.

According to one or more embodiments, the liquid may further include a radical inhibitor. The radical inhibitor may be at least one of 2,4-Dimethyl-6-tert-butylphenol, 2,2,6,6-Tetramethylpiperidin-1-yl)oxyl (TEMPO), Dibenzylhydroxylamine (DBHA), Diethylhydroxylamine (DEHA), 2,5-Di-tert-butylhydroquinone (DTBHQ), moquinol (MEHQ), butylated hydoxytoluene (BHT), Hydroquinone (HQ), Para-benzoquinone (p-Bq), Ortho-benzoquinone (o-Bq), N, N-Dimethylisopropanolamine (DMPA), Phenothiazine (PTZ), 2,4-dinotrophenol (DNP), 2,4-dinitro-6-sec-butylphenol (DNBP), 2,6-dinitro-p-cresol, 1,4-Phenylenediamine, or N,N-dimethylaniline (DMA). The radical inhibitor may inhibit self-reaction of the liquid due to aging and changes in temperature. According to one or more embodiments, the liquid in the liquid-powder reliner may include up to 0.5% by weight of the radical inhibitor. Preferably, the liquid in the liquid-powder reliner may include 0.1% by weight of the radical inhibitor.

According to one or more embodiments, dimethylparatoluidine may be used as an amine accelerator. According to one or more embodiments, the amine accelerator may also be at least one of benzyl dimethylamine (BDMA), tris(dimethylaminomethyl) phenol (DMP-30), nonylphenol, benzyl alcohol, or triethanolamine (TEA). The amine accelerator may be configured to allow for the polymerization reaction of the acrylic monomer to occur more quickly at a lower temperature. The lower temperature may be a room temperature or a body temperature. According to one or more embodiments, the liquid in the liquid-powder reliner may include between 0.25% to 5% by weight of amine accelerator. If there is less than 0.25% of the amine accelerator in the liquid of the liquid-powder reliner, the acrylic monomer may not react quickly enough or may not react at all at the lower temperature. If there is more than 5% of the amine accelerator in the liquid of the liquid-powder reliner, the acrylic monomer may react too quickly at the lower temperature such that the setting time of the reliner after the liquid and the powder are mixed may occur too quickly. Furthermore, if there is more than 5% of the amine accelerator in the liquid of the liquid-powder reliner, a shelf life of the liquid may be shortened due to a possible reaction of the acrylic monomer before the liquid and the powder are mixed.

### [Description of the Powder]

According to one or more embodiments, the powder may include a methacrylate polymer or co-polymer, an initiator (e.g. radical initiator), an anticaking agent, and coloring agents. The powder is not limited to the materials listed hereto. Before the liquid and the powder are mixed, the powder may be a fine powder and pink in color, but is not limited thereto. According to one or more embodiments, the powder may be stored in a temperature of 20-25° C., but is not limited thereto.

According to one or more embodiments, the methacrylate polymer or co-polymer of the powder may be polyethyl methacrylate. According to one or more embodiments, the methacrylate polymer or co-polymer of the powder may be at least one of methyl methacrylate, ethyl methacrylate, t-butyl methacrylate, n-butyl methacrylate, lauryl methacrylate, dodecyl methacrylate, Stearyl methacrylate, cyclohexyl methacrylate, benzyl methacrylate, isobonyl methacrylate, isopropyl methacrylate, 2-hydroxyethyl methacrylate, 3-hydroxypropyl methacrylate, 2-hydroxy-1,3-dimethacryloxypropane, isobutyl meth acrylate, butoxyethyl meth acrylate, hydroxypropyl methacrylate, tetrahydrofurfuryl meth acrylate, glycidyl methacrylate, 2-methoxyethyl meth acrylate, 2-ethylhexyl methacrylate, benzyl methacrylate; or methacrylate/styrene copolymers. According to one or more embodiments, the methacrylate polymer or co-polymer of the powder may be swelled by the aliphatic acid ester-based plasticizer, the acrylic monomer, and the solvent of the liquid. A resin material of the soft relining material may be formed from the swelling. According to one or more embodiments, the powder in the liquid-powder reliner may include 70% to 100% by weight of the methacrylate polymer or co-polymer. If there is less than 70% of the methacrylate polymer or co-polymer in the powder, the material of the liquid-powder reliner, after the liquid and the powder are mixed and the reliner is cured, may not have a sufficient elasticity.

According to one or more embodiments, benzoyl peroxide can be used as the radical initiator. According to one or more embodiments, the powder in the liquid-powder reliner may include less than 1% by weight of the radical initiator. According to one or more embodiments, other organic peroxides can be used as the radical initiator such as methyl ethyl ketone peroxide or peroxyacetic acid. If there is more than 1% of the radical initiator in the powder, the setting time of the reliner once the liquid and the powder are mixed may be too fast due to excessive heat generation and the coloring of the reliner may be too yellowish.

According to one or more, the anticaking agent may be silica. According to one or more embodiments, the anticaking agent may also be magnesium stearate or talcum powder. The anticaking agent may be configured to reduce clumping in the powder before the liquid and the powder are mixed. According to one or more embodiments, the powder in the liquid-powder reliner may include less than 10% by weight of the radical initiator. If there is more than 10% of the anticaking agent in the powder, the reliner, once the liquid and the powder are mixed, may be too hard (i.e. not soft enough) and the coloring the reliner may be opaque. An opaque color of the reliner is not preferred due to aesthetic effects.

The coloring agent may be optional. According to one or more embodiments, the coloring agent may be at least one of rayon fibers or coloring pigments. The coloring agent may be configured to make the color of the soft relining material match the color of the dentures such that one cannot see the difference between the soft relining material and the base of the dentures. The coloring agent may account for less than 2% by weight of the powder.

### [Creation and Application of the Liquid-Powder Reliner]

According to one or more embodiments, the soft relining material may be produced by mixing the liquid and the powder. According to one or more embodiments, 8 grams of liquid and 11 grams of powder may be mixed in a mixing cup for 30 seconds, however the amounts and the timing is not limited thereto. After the liquid and the powder is mixed for 30 seconds, a slurry may be produced. The slurry, after some time, may harden, and form the liquid-powder reliner which may be applied to a base 3D printed dentures made of resin and a base dentures made of acrylic material. The base of the dentures to which the slurry is applied may be a side of the dentures that contacts a wearer's roof of their mouth.

According to one or more embodiments, the liquid-powder reliner may be soft and have a Shore A0 hardness greater than 30 and less than or equal to 50 (i.e. 30<Shore A0≤50). According to another embodiment, after the liquid and the powder are mixed, the slurry may be extra soft and have a Shore A0 hardness less than or equal to 30 (i.e. Shore A0≤30).

According to one or more embodiments, the consistency of the flow (measured by the diameter (d) in millimeters) may be medium and have a diameter greater than or equal to 25 mm and less than 60 mm (i.e. 25≤d<60). According to another embodiment, the consistency of the flow may be high and have a diameter greater than or equal to 60 mm and less than 100 mm (i.e. 60≤d<100).

According to one or more embodiments, before the slurry is applied to the dentures, a bonding agent may be applied to the base of the dentures. The bonding agent may comprise an acrylic polymer in which the acrylic polymer chains adhere to the denture base. The bonding agent may air-dry after it is applied to the base of the dentures. In a state in which the dentures are 3D printed dentures having a resin composite base made of multi-functional (i.e. dimethacrylate) resin, the acrylic polymer chains may dissolve the surface of the multi-functional (i.e. dimethacrylate) resin polymer and the acrylic polymer will penetrate and entangle with the multi-functional (i.e. dimethacrylate) resin polymer, and allow the slurry mixture of the liquid-powder reliner to adhere to the base of the 3D printed denture base. The acrylic monomer of the liquid-powder reliner may penetrate the dissolved surface of the 3D printed dentures having a resin composite base made of multi-functional (i.e. dimethacrylate) resin and entangle with the penetrated polymer of the bonding agent. The bonding agent may have a similar or same effect on acrylic base dentures as it does on 3D printed dentures having a resin composite base made of multi-functional (i.e. dimethacrylate) resin.

Because the slurry is applied to the dentures that have been treated with the bonding agent, the slurry may cure with a strong bond to the dentures. After several minutes, the slurry bonded to the dentures may cure to a soft rubbery state adhered to the denture. The slurry may provide a soft cushioning between the roof of the wearer's mouth and the base of the dentures, while retaining its shape for tight contact between the denture and the mouth.

According to one or more embodiments, the liquid and the powder may be manually mixed or mixed by a machine.

### Additional Aspects

The one or more embodiments described above may encompass the concepts as described.

### [Configuration 1]

A composition for a soft relining material includes:
10 to 70% by weight of a methacrylate monomer having at least one unsaturated double bond;
30 to 80% by weight of a plasticizer; 5% to 20% by weight of a solvent;
up to 2% by weight of a stabilizer;
up to 0.5% by weight of an inhibitor; and
0.25 to 5% by weight of an accelerator.

### [Configuration 2]

In the soft relining material according to configuration 1, the plasticizer includes at least one of of dibutyl phthalate, acetyl tributyl citrate, benzyl salicylate, trimelitic acid esters, fatty acid esters, dicarboxylic acid esters, acetic acid esters, maleic acid esters, fumaric acid esters, and citric acid esters.

### [Configuration 3]

In the soft relining material according to configuration 1, the plasticizer includes a mixture of acetyl tributyl citrate and benzyl salicylate.

### [Configuration 4]

In the soft relining material according to configuration 1, the plasticizer includes acetyl tributyl citrate.

### [Configuration 5]

In the soft relining material according to configuration 1, the plasticizer includes benzyl salicylate.

### [Configuration 6]

In the soft relining material according to any one of configurations 1 to 5, the plasticizer is 65% by weight.

### [Configuration 7]

In the soft relining material according to any one of configurations 1 to 6, the solvent is ethanol.

### [Configuration 8]

In the soft relining material according to any one of configurations 1 to 7, the stabilizer is 2,4-dihydroxybenzophenone.

### [Configuration 9]

In the soft relining material according to any one of configurations 1 to 8, the stabilizer is an ultra violet (UV) stabilizer.

### [Configuration 10]

In the soft relining material according to any one of configurations 1 to 9, the stabilizer is 1% by weight.

### [Configuration 11]

In the soft relining material according to any one of configurations 1 to 10, the inhibitor is moquinol (MEHQ).

### [Configuration 12]

In the soft relining material according to any one of configurations 1 to 10, the inhibitor is butylated hydoxytoluene (BHT).

### [Configuration 13]

In the soft relining material according to any one of configurations 1 to 12, the inhibitor is 0.1% by weight.

### [Configuration 14]

In the soft relining material according to any one of configurations 1 to 13, the accelerator is dimethylparatoluidine.

It should be understood that the embodiments disclosed herein is illustrative and non-restrictive in every respect. The scope of the present invention is defined by the terms of the claims rather than the description above and is intended to include any modifications within the scope and meaning equivalent to the terms of the claims

What is claimed is:

1. A composition for a soft relining material, comprising:
10 to 70% by weight of a methacrylate monomer having at least one unsaturated double bond;
30 to 80% by weight of a plasticizer;
5% to 20% by weight of a solvent;
up to 2% by weight of a stabilizer;
up to 0.5% by weight of an inhibitor; and
0.25 to 5% by weight of an accelerator.

2. The composition according to claim 1, wherein the plasticizer comprises at least one of of dibutyl phthalate, acetyl tributyl citrate, benzyl salicylate, trimelitic acid esters, fatty acid esters, dicarboxylic acid esters, acetic acid esters, maleic acid esters, fumaric acid esters, and citric acid esters.

3. The composition according to claim 1, wherein the plasticizer comprises a mixture of acetyl tributyl citrate and benzyl salicylate.

4. The composition according to claim 1, wherein the plasticizer comprises acetyl tributyl citrate.

5. The composition according to claim 1, wherein the plasticizer comprises benzyl salicylate.

6. The composition according to claim 1, wherein the plasticizer is 65% by weight.

7. The composition according to claim 1, wherein the solvent is ethanol.

8. The composition according to claim 1, wherein the stabilizer is 2,4-dihydroxybenzophenone.

9. The composition according to claim 1, wherein the stabilizer is an ultra violet (UV) stabilizer.

10. The composition according to claim 1, wherein the stabilizer is 1% by weight.

11. The composition according to claim 1, wherein the inhibitor is moquinol (MEHQ).

12. The composition according to claim 1, wherein the inhibitor is butylated hydoxytoluene (BHT).

13. The composition according to claim 1, wherein the inhibitor is 0.1% by weight.

14. The composition according to claim 1, wherein the accelerator is dimethylparatoluidine.

15. A soft relining material, comprising:
a powder; and
a liquid comprising:
10 to 70% by weight of a methacrylate monomer having at least one unsaturated double bond;
30 to 80% by weight of a plasticizer;
5% to 20% by weight of a solvent;
up to 2% by weight of a stabilizer;
up to 0.5% by weight of an inhibitor; and
0.25 to 5% by weight of an accelerator.
